(19) United States
(12) Patent Application Publication
(10) Pub. No.: US 2024/0122857 A1
(43) Pub. Date: Apr. 18, 2024

(54) PHARMACEUTICAL FORMULATIONS OF INDOLEAMINE 2, 3-DIOXYGENASE INHIBITORS

(71) Applicant: BRISTOL-MYERS SQUIBB COMPANY, Princeton, NJ (US)

(72) Inventors: Jing TAO, Princeton, NJ (US); Anisha PATEL, Princeton, NJ (US); Umesh KESTUR, Princeton, NJ (US); Admassu ABEBE, Princeton, NJ (US); Divyakant Shantilal DESAI, Princeton, NJ (US)

(21) Appl. No.: 17/753,657

(22) PCT Filed: Sep. 10, 2020
(86) PCT No.: PCT/US2020/050085

§ 371 Date: Mar. 10, 2022

Related U.S. Application Data
(60) Provisional application No. 62/898,855, filed on Sep. 11, 2019.

(51) Int. Cl. A61K 9/20; A61K 31/47; A61K 47/02; A61K 47/12; A61K 47/26; A61K 47/32; A61K 47/38

(52) U.S. Cl. CPC A61K 9/2077; A61K 31/47; A61K 47/02; A61K 47/12; A61K 47/26; A61K 47/32; A61K 47/38

(57) ABSTRACT

The present application is directed to a pharmaceutical composition comprising (R)—N-(4-chlorophenyl)-2-((1S, 4S)-4-(6-fluoroquinolin-4-yl) cyclohexyl) propanamide methane sulfonic acid salt that is resistant to salt disproportionation:

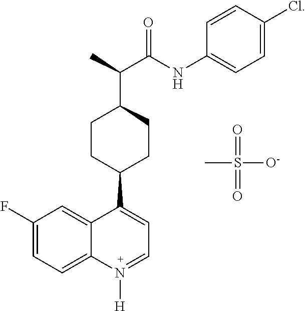

### CROSS-REFERENCE TO RELATED APPLICATIONS

This application is the national stage filing under USC 371 of International Application No. PCT/US2020/050085, filed Sep. 10, 2020, which claims the benefit of U.S. Provisional Application No. 62/898,855, filed Sep. 11, 2019, which are incorporated herein by reference in their entirety for all purposes.

### TECHNICAL FIELD

The present application is directed to a pharmaceutical composition comprising (R)—N-(4-chlorophenyl)-2-((1S, 4S)-4-(6-fluoroquinolin-4-yl) cyclohexyl) propanamide methane sulfonic acid salt that has low salt disproportionation resulting in a stable solid dosage form.

### BACKGROUND

Indole amine 2,3-dioxygenase (“IDO” or “IDO1”) is an IFN-γ target gene that plays a role in immunomodulation, and its immunosuppressive function manifests in several manners. A pathophysiological link exists between IDO and cancer. Disruption of immune homeostasis is intimately involved with tumor growth and progression, and the production of IDO in the tumor microenvironment appears to aid in tumor growth and metastasis. Moreover, increased levels of IDO activity are associated with a variety of different tumors (Brandacher, G. et al., Clin. Cancer Res., 12(4):1144-1151 (Feb. 15, 2006)). In addition to cancer, IDO has been implicated in, among other conditions, immunosuppression, chronic infections, and autoimmune diseases or disorders (e.g., rheumatoid arthritis).

(R)—N-(4-chlorophenyl)-2-((1S, 4S)-4-(6-fluoroquinolin-4-yl) cyclohexyl) propanamide (“Compound I,”) also generally referred to as “linrodostat,” was disclosed as a potent inhibitor of IDO (see, e.g., International Publication No. WO2016/073770). The methane sulfonic acid salt of Compound I (“Compound I-MSA”) was disclosed as the salt form with superior properties.

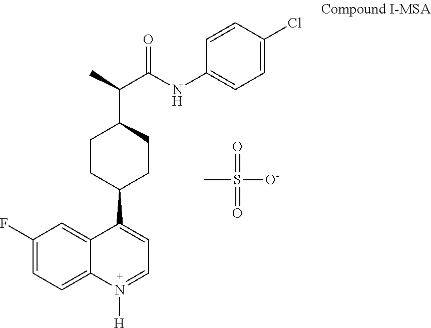

One of the most important aspects in pharmaceutical formulation development is the identity and combination of excipients and how they interact with the active pharmaceutical ingredient (“API”). Many APIs are manufactured and formulated as salts, due to improved solid state properties leading to improved dissolution rates and bioavailability over the free form crystalline API forms. These free forms of the API may have a basic site where the pKa is too low (e.g., pKa is 4.6) and risk encountering long term storage stability problems, proton transfer, and/or further disproportionation.

In the development of solid oral dosage forms containing the salt of the ionizable drug, some excipients are known to cause conversion of the API to the free base. The design of the formulation must take into account the factors affecting salt disproportion during processing or storage and how this impacts product quality and performance. Thus, there is a need for stable pharmaceutical compositions.

### SUMMARY

Described herein are pharmaceutical compositions of Compound I-MSA suitable for oral administration.

In a first aspect, the invention provides a pharmaceutical composition suitable for oral administration comprising:
(i) a therapeutically effective amount of (R)—N-(4-chlorophenyl)-2-((1S, 4S)-4-(6-fluoroquinolin-4-yl) cyclohexyl) propanamide methane sulfonic acid salt present in an amount between 5% to 40% w/w of the composition having the structure:

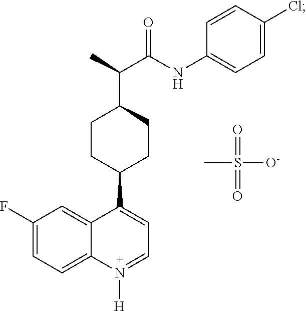

(ii) crospovidone as a disintegrant present in an amount between 2.0% to 7.0% w/w of the composition; and,
(iii) magnesium stearate as a lubricant present in amount between 0.25% to 1.75% w/w of the composition;
wherein the ratio of (R)—N-(4-chlorophenyl)-2-((1S, 4S)-4-(6-fluoroquinolin-4-yl) cyclohexyl) propanamide methane sulfonic acid salt to total magnesium stearate is 8.0 to 40.0 by weight; and,
wherein salt disproportionation of (R)—N-(4-chlorophenyl)-2-((1S, 4S)-4-(6-fluoroquinolin-4-yl) cyclohexyl) propanamide methane sulfonic acid to (R)—N-(4-chlorophenyl)-2-((1S,4S)-4-(6-fluoroquinolin-4-yl) cyclohexyl) propanamide is less than 25% by weight.

In an embodiment, the pharmaceutical composition further comprises microcrystalline cellulose as a first diluent and lactose anhydrous as a second diluent present in a total amount between 50% to 80% w/w of the composition.

In an embodiment, the pharmaceutical composition further comprises silicon dioxide as a glidant present in an amount of 1.0% to 3.0% w/w of the composition.

In an embodiment, the pharmaceutical composition comprises salt disproportionation of (R)—N-(4-chlorophenyl)-2-((1S, 4S)-4-(6-fluoroquinolin-4-yl) cyclohexyl) propanamide methane sulfonic acid salt to (R)—N-(4-chlorophenyl)-2-((1S, 4S)-4-(6-fluoroquinolin-4-yl)cyclohexyl) propanamide in amount of less than 5% by weight. In another embodiment, the pharmaceutical composition comprises salt disproportionation of (R)—N-(4-chlorophenyl)-2-((1S, 4S)-4-(6-fluoroquinolin-4-yl)cyclohexyl)propanamide methane sulfonic acid salt to (R)—N-(4-chlorophenyl)-2-((1S, 4S)-4-(6-fluoroquinolin-4-yl)cyclohexyl)propanamide in amount of less than 3% by weight.

In an embodiment, the pharmaceutical composition comprises (R)—N-(4-chlorophenyl)-2-((1S,4S)-4-(6-fluoroquinolin-4-yl)cyclohexyl)propanamide methane sulfonic acid salt to total magnesium stearate in a ratio of 23.0 to 40.0 by weight.

In an embodiment, the pharmaceutical composition comprises (R)—N-(4-chlorophenyl)-2-((1S, 4S)-4-(6-fluoroquinolin-4-yl)cyclohexyl)propanamide methane sulfonic acid salt present in an amount between 15% to 20% w/w of the composition.

In an embodiment, the pharmaceutical composition comprises a first diluent and a second diluent in a ratio ranging from 2:1 to 1:2 by weight.

In another embodiment, the pharmaceutical composition comprises a first diluent in an amount ranging from 25% to 40% w/w of the composition. In a further embodiment, the pharmaceutical composition comprises a second diluent present an amount ranging from 25% to 40% w/w of the composition.

In an embodiment, the pharmaceutical composition comprises silicon dioxide present in an amount of 2.0% w/w of the composition.

In an embodiment, the pharmaceutical composition comprises an intra-granular phase and an extra-granular phase. In a further embodiment, the pharmaceutical composition comprises:
(a) an intra-granular phase comprising:
   (i) (R)—N-(4-chlorophenyl)-2-((1S, 4S)-4-(6-fluoroquinolin-4-yl) cyclohexyl) propanamide methane sulfonic acid salt present in an amount between 12% to 18% w/w of the composition;
   (ii) crospovidone as a disintegrant present in an amount of 2% to 3% w/w of the composition;
   (iii) magnesium stearate as a lubricant present in an amount of 0.25% to 0.75% w/w of the composition;
(b) an extra-granular phase comprising:
   (i) crospovidone as a disintegrant present in an amount of 2% to 3% w/w of the composition; and,
   (ii) magnesium stearate as a lubricant present in an amount of 0.50% to 1.00% w/w of the composition.

In an embodiment, the pharmaceutical composition comprises an intra-granular phase further comprising microcrystalline cellulose as a first diluent and lactose anhydrous as a second diluent present in a total amount between 75% to 80% w/w of the composition; and, silicon dioxide as a glidant present in an amount of 1.5% to 2.5% w/w of the composition.

In an embodiment, the pharmaceutical composition comprises salt disproportionation of (R)—N-(4-chlorophenyl)-2-((1S,4S)-4-(6-fluoroquinolin-4-yl)cyclohexyl)-propanamide methane sulfonic acid salt to (R)—N-(4-chlorophenyl)-2-((1S,4S)-4-(6-fluoroquinolin-4-yl)cyclohexyl)propanamide in amount of less than 10% after 12 weeks at 40° C. and 75% open relative humidity and has a particle range distribution characterized by a D90 having a value from about 7 microns to about 165 microns.

In an embodiment, the pharmaceutical composition comprises salt disproportionation of (R)—N-(4-chlorophenyl)-2-((1S, 4S)-4-(6-fluoroquinolin-4-yl) cyclohexyl) propanamide methane sulfonic acid salt to (R)—N-(4-chlorophenyl)-2-((1S, 4S)-4-(6-fluoroquinolin-4-yl)cyclohexyl) propanamide in amount of less than 3% by weight after 24 weeks stored in a 200 cc high-density polyethylene bottle at 25° C. and 60% relative humidity.

In an embodiment, the pharmaceutical composition comprises a particle range distribution characterized by a D90 having a value from about 10 microns to about 165 microns.

In an embodiment, the pharmaceutical composition comprises salt disproportionation of (R)—N-(4-chlorophenyl)-2-((1S,4S)-4-(6-fluoroquinolin-4-yl) cyclohexyl)-propanamide methane sulfonic acid salt to (R)—N-(4-chlorophenyl)-2-((1S,4S)-4-(6-fluoroquinolin-4-yl)cyclohexyl)propanamide in amount of less than 3% by weight after 6 months at 25° C. and 60% relative humidity with blister packaging.

In an embodiment, the pharmaceutical composition comprises salt disproportionation of (R)—N-(4-chlorophenyl)-2-((1S,4S)-4-(6-fluoroquinolin-4-yl)cyclohexyl)-propanamide methane sulfonic acid salt to (R)—N-(4-chlorophenyl)-2-((1S,4S)-4-(6-fluoroquinolin-4-yl)cyclohexyl)propanamide in amount of less than 3% by weight after 4 weeks at 25° C. and 60% relative humidity.

In an embodiment, the pharmaceutical composition comprises salt disproportionation of (R)—N-(4-chlorophenyl)-2-((1S,4S)-4-(6-fluoroquinolin-4-yl)cyclohexyl)-propanamide methane sulfonic acid salt to (R)—N-(4-chlorophenyl)-2-((1S,4S)-4-(6-fluoroquinolin-4-yl)cyclohexyl)propanamide in amount of less than 3% by weight after 4 weeks at 40° C. and 75% relative humidity.

In an embodiment, the pharmaceutical composition comprises a blend and the salt disproportionation of (R)—N-(4-chlorophenyl)-2-((1S,4S)-4-(6-fluoroquinolin-4-yl)cyclohexyl)propanamide methane sulfonic acid to (R)—N-(4-chlorophenyl)-2-((1S,4S)-4-(6-fluoroquinolin-4-yl)cyclohexyl)propanamide is less than 3% by weight after 24 weeks at 25° C. and 60% relative humidity.

In an embodiment, the pharmaceutical composition comprises a composition selected from the group consisting of tablet, crushed tablet, capsule or sprinkled contents from a capsule, mini-tablets, and beads.

In another embodiment, the pharmaceutical composition further comprises citric acid.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1 illustrates the percentage of free base in tablet formulations containing croscarmellose sodium or crospovidone as the disintegrant at 40° C./75% relative humidity (“RH”), open high-density polyethylene (“HDPE”) bottle.

FIG. 2 illustrates the percentage of free base in tablet formulations of different drug load to magnesium stearate ratios at 40° C./75%, open high-density polyethylene bottle.

FIG. 3 illustrates an embodiment of the method of producing a 100 mg, 50 mg, and 25 mg film-coated tablet of Compound I-MSA.

### DETAILED DESCRIPTION

The present invention may be understood more readily by reference to the following detailed description taken in connection with the accompanying figures and examples, which form a part of this invention. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention. Also, as used in the specification including the appended claims, the singular forms “a,” “an,” and “the” include the plural, and reference to a particular numerical value includes at least that particular value, unless the context clearly dictates otherwise.

The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated compounds, which allows the presence of only the named compounds, along with any pharmaceutically carriers, and excludes other compounds.

All ranges disclosed herein are inclusive of the recited endpoint and independently combinable (for example, the range of “from 100 mg to 200 mg” is inclusive of the endpoints, 100 mg and 200 mg, and all the intermediate values). The endpoints of the ranges and any values disclosed herein are not limited to the precise range or value; they are sufficiently imprecise to include values approximating these ranges and/or values.

As used herein, “pharmaceutically acceptable salts” refer to derivatives of the disclosed compounds wherein the parent compound is modified by making acid or base salts thereof.

The term “API” refers to the active pharmaceutical ingredient. As used herein, API refers to Compound I-MSA or (R)—N-(4-chlorophenyl)-2-((1S, 4S)-4-(6-fluoroquinolin-4-yl) cyclohexyl)propanamide methane sulfonic acid salt.

### Pharmaceutically Acceptable Compositions and Formulations

In aspects of the application, the pharmaceutical compositions of the invention include from 10% to 40% w/w of the API, based on the weight of the pharmaceutical composition. In another embodiment, the pharmaceutical compositions of the invention include from 15% to 20% w/w of the API, based on the weight of the pharmaceutical composition. In another embodiment, the pharmaceutical compositions of the invention include from 17% to 18% w/w of the API, based on the weight of the pharmaceutical composition.

The pharmaceutical compositions of the invention include a diluent. The diluent of the invention can include, for example, a first diluent, and optionally, a second diluent. Diluents generally known in the art include, for example, sugar alcohols, sugars, celluloses, starch-derived diluents, and combinations thereof. More specific diluents known in the art include dextrin, sucrose, sorbitol, sodium saccharin, acesulfame potassium, xylitol, aspartame, mannitol, starch, cornstarch, PVP (polyvinyl pyrrolidone), low molecular weight HPC (hydroxypropyl cellulose), microcrystalline cellulose (“MCC”), low molecular weight HPMC (hydroxypropyl methylcellulose), low molecular weight carboxymethyl cellulose, ethyl cellulose, dicalcium phosphate, silicified microcrystalline cellulose, alginates, gelatin, polyethylene oxide, acacia, dextrin, sucrose, magnesium aluminum silicate, and polymethacrylates. An embodiment of a diluent of the present application is lactose, for example lactose (anhydrous), high velocity lactose, or a combination thereof. Another embodiment is microcrystalline cellulose, for example, microcrystalline cellulose PH 302. The present application contemplates the use of a combination of diluents, such as microcrystalline cellulose and lactose.

In those aspects of the invention including two diluents, that is, a first diluent and a second diluent, the ratio of the first diluent to the second diluent is between 2:1 and 1:2. In an embodiment, the ratio of the first diluent to the second diluent is 1:1. In an embodiment, the first diluent is microcrystalline cellulose and the second diluent is lactose.

The compositions of the invention include between 50% and 80% w/w of diluent, based on the weight of the pharmaceutical composition. In an embodiment, the pharmaceutical composition comprises between 75% and 80% w/w of diluent, based on the weight of the pharmaceutical composition. In an embodiment, the pharmaceutical composition comprises between 35% and 40% w/w of a first diluent and between 35% and 40% w/w of a second diluent, based on the weight of the pharmaceutical composition.

The pharmaceutical compositions of the invention may include a glidant. Glidants known in the art may include, but are not limited to, silicon dioxide, colloidal silicon dioxide, talc, magnesium carbonate, calcium silicate, fumed silicon dioxide, starch, and combinations thereof. The present application contemplates the use of silicon dioxide as a glidant. The compositions of the invention include between 1.0% and 3.0% w/w of a glidant, based on the weight of the pharmaceutical composition. In an embodiment, the pharmaceutical compositions comprises between 1.75% and 2.25% w/w of a glidant, based on the weight of the pharmaceutical composition.

In an embodiment of the invention, the pharmaceutical composition comprises granules. In an embodiment, the granules of the composition can have an intra-granular phase and an extra-granular phase. In an embodiment, the intra-granular phase comprises a glidant, with no glidant being in the extra-granular phase.

The pharmaceutical compositions of the invention includes a disintegrant. Disintegrants are known in the art include, for example, starch-based disintegrants, cellulose-based disintegrants, povidone-based disintegrants, and the like. Specific examples of disintegrants include, but are not limited to, calcium carbonate, potato or tapioca starch, alginic acid, certain silicates, sodium carbonate, crospovidone (cross-linked polyvinylpyrrolidone (“PVP”)), sodium carboxymethyl starch (sodium starch glycolate), cross-linked sodium carboxymethyl cellulose (croscarmellose), pre-gelatinized starch (starch 1500), microcrystalline starch, water insoluble starch, sodium alginate, calcium carboxymethyl cellulose, and magnesium aluminum silicate (Veegum). The present application contemplates the use of crospovidone (a cross-linked povidone) as a disintegrant.

The pharmaceutical compositions of the invention include between 2.0% and 7.0% w/w of a disintegrant, based on the weight of the pharmaceutical composition. In an embodiment, the pharmaceutical compositions of the invention include 2.5% w/w of a disintegrant in an intra-granular phase and 2.5% w/w of a disintegrant in an extra-granular phase, based on the weight of the pharmaceutical composition.

The pharmaceutical compositions of the invention may include a lubricant. Lubricants are known in the art and include, for example, stearic acid, stearic acid salts, and combinations thereof, and the like. Examples of stearic acid salts are calcium stearate, magnesium stearate, sodium stearyl fumarate, and combinations thereof. The lubricant of the invention can include one lubricant or can include a combination of (i.e., more than one) lubricants. The present application contemplates the use of magnesium stearate as a lubricant.

The pharmaceutical compositions of the invention include between about 0.25% and about 1.75% w/w of a lubricant. In an embodiment a lubricant forms part of the intra-granular phase and part of the extra-granular phase. In an embodiment, the pharmaceutical compositions of the invention include between 0.25% and 0.75% w/w of a lubricant in an intra-granular phase, based on the weight of the pharmaceutical composition. In an embodiment, the pharmaceutical compositions of the invention include between 0.50% and 1.00% w/w of a lubricant in an extra-granular phase, based on the weight of the pharmaceutical composition.

Provided compositions may be formulated into a unit dosage form. Such formulations are well known to one of ordinary skill in the art. In certain embodiments, the present invention provides a formulation comprising a solid dosage form as a tablet, crushed tablet, capsule or sprinkled contents from a capsule, mini-tablets, or beads.

The pharmaceutical compositions of the invention may include an organic acid. The present application contemplates the use of citric acid as an organic acid.

### Tablet Preparation

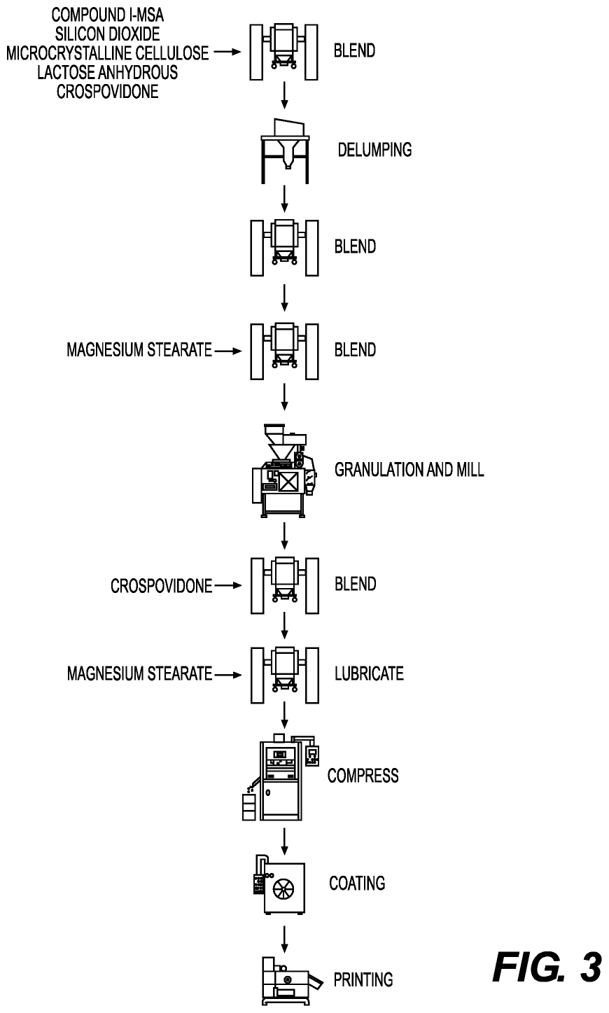

Tablets may be prepared according to methods known in the art, including dry granulation (e.g., roller compaction), wet granulation (e.g., fluid bed granulation and high shear granulation), and direct compression, and the type of excipients used will vary accordingly. The present application is directed to a method of preparing tablets via dry granulation (see, e.g., FIG. 3).

For example, the tablets were prepared according to the following general steps which are also illustrated in FIG. 3:
(1) Pre-blend: the API and pharmaceutically acceptable excipients were blended during the manufacturing process. In one non-limiting example, first, the API and intra-granular excipients (first diluent, optional second diluent, glidant, disintegrant; except for the intra-granular lubricant) were sifted through a screen, added to a blender, and blended for a first blending time period to produce an initial blend. Separately, an intra-granular lubricant was passed through a screen, mixed with a portion of the initial blend, added to the blender, and blended for a second blending time period.
(2) Dry Granulation: (a) roller compaction: the API and pharmaceutically acceptable excipients were passed through a roller compactor to produce compacts. Compacts were then milled to achieve granules. (b) milling (preparation of milled/sifted granule): the API and pharmaceutically acceptable excipients were milled and/or sifted.
(3) Extra-granular blending: granules comprising the API and intra-granular excipients that had been milled/sifted were blended with extra-granular excipients in a final blending.
(4) Compressing: The final blend was compressed into tablets using a tablet press.
(5) Optionally, tablets were film-coated with a film-coating agent.

### EXAMPLES

The following examples are offered for purposes of illustration, and are not intended to limit the scope of the claims provided herein. All literature citations in these examples and throughout this specification are incorporated herein by references for all legal purposes to be served thereby.

The present application provides a pharmaceutical composition comprising the methane sulfonic acid salt of Compound I. The chemical formula for (R)—N-(4-chlorophenyl)-2-((1S, 4S)-4-(6-fluoroquinolin-4-yl)cyclohexyl)propanamide, also referred to as “Compound I,” “linrodostat” or the “free base,” is C₂₅H₂₈ClFN₂O₄S, which has a molecular weight of 410.92 g/mol and the molecular weight of the methane sulfonic acid salt is 507.02 g/mol. To achieve a drug load of 12.5% as the free base, 15.43% of the drug substance as the methane sulfonic acid salt must be added in the formulation.

### Preliminary Tablet Formulation Development

In an excipient compatibility study, Compound I-MSA exhibited acceptable chemical stability with commonly used pharmaceutical excipients indicating that Compound I-MSA was amenable to formulation with the excipients.

### Example 1: Type of Disintegrant

Raman imaging detected free base in tablets that were subjected to stress storage conditions at 40° C./75% relative humidity (“RH”) open bottle, suggesting that salt disproportionation had occurred to Compound I-MSA. Following this observation, several factors were evaluated to overcome the occurrence of salt disproportionation of the methane sulfonic acid salt to the free base. One factor with a surprising impact was the type of disintegrant.

Cations such as Na⁺, Ca²⁺, or Mg²⁺ were found to facilitate or induce disproportionation of the methane sulfonic acid salt. In the development of the formulation, a source of sodium ions that was suspected as a contributing factor in the methane sulfonic acid salt disproportionation was croscarmellose sodium. Consequently, crospovidone was evaluated as an alternate disintegrant in the tablet formulation. Tables 1 and 2 show the tablet formulations made with crospovidone.

TABLE 1
Compositions with Crospovidone

| | | | | |
|---|---|---|---|---|
| | | | mg in | mg in |
| | | Quantity per Tablet | 50 mg | 25 mg |
| | | mg in 100 mg | (free | (free |
| | % | (free base) | base) | base) |
| Component | w/w | tablets | tablets | tablets |

Intra-granular

| | | | | |
|---|---|---|---|---|
| Compound I-MSA | 15.425^{a} | 123.36 | 61.68 | 30.84 |
| Lactose Anhydrous | 38.165 | 305.32 | 152.66 | 76.33 |
| Microcrystalline | 38.165 | 305.32 | 152.66 | 76.33 |
| Cellulose | | | | |
| Silicon Dioxide | 2.00 | 16.0 | 8.0 | 4.0 |
| Crospovidone | 2.50 | 20.0 | 10.0 | 5.0 |
| Magnesium Stearate | 0.50 | 4.0 | 2.0 | 1.0 |

Extra-granular

| | | | | |
|---|---|---|---|---|
| Crospovidone | 2.50 | 20.0 | 10.0 | 5.0 |
| Magnesium Stearate | 0.75 | 6.0 | 3.0 | 1.5 |
| Total | 100.0 | 800.0 | 400.0 | 200.0 |
| Film Coat | 3.0 | 24.0 | | |
| Film Coat | 3.8 | | 15.2 | |
| Film Coat | 4.6 | 24.0 | | 9.20 |
| Total | | 824.0 | 415.2 | 209.20 |

Table 1 illustrates the components of the pharmaceutical composition, including the function of the component as well as the % w/w of the composition. The pharmaceutical composition was formulated in 100 mg, 50 mg, and 25 mg tablets as shown in the table.

TABLE 2
Compositions Containing Crospovidone with either
Magnesium Stearate or Stearic Acid as Lubricant

| | | | | |
|---|---|---|---|---|
| | | Quantity per Tablet | | mg in |
| | % | mg in 100 mg | % | 100 mg |
| Component | w/w | tablets | w/w | tablets |

Intra-granular

| | | | | |
|---|---|---|---|---|
| Compound I-MSA | 15.42^{a} | 123.36 | 15.42^{a} | 123.36 |
| Lactose Anhydrous | 38.165 | 305.32 | 38.04 | 304.32 |
| Microcrystalline | 38.165 | 305.32 | 38.04 | 304.32 |
| Cellulose | | | | |
| Silicon Dioxide | 2.00 | 16.00 | 2.00 | 16.00 |
| Crospovidone | 2.50 | 20.0 | 2.50 | 20.00 |
| Magnesium Stearate | 0.50 | 4.00 | NA | NA |
| Stearic acid | NA | NA | 1.50 | 12.00 |

Extra-granular

| | | | | |
|---|---|---|---|---|
| Crospovidone | 2.50 | 20.0 | 2.50 | 20.00 |
| Magnesium Stearate | 0.75 | 6.00 | NA | NA |
| Stearic Acid | | | 1.00 | 8.00 |
| Total | 100.00 | 800.00 | 100.00 | 800.00 |

^{a}Strength as free base.

Table 2 illustrates different tablet compositions (or formulations) containing crospovidone with either magnesium stearate or stearic acid as a lubricant in 100 mg tablets.

TABLE 3
Tablet Composition with Corscarmellose Sodium (Comparative)

| | | | |
|---|---|---|---|
| | % | mg in 100 mg | mg in 25 |
| Component | w/w | tablets | mg tablet |

Intra-granular

| | | | |
|---|---|---|---|
| Compound I-MSA | 15.42^{a} | 123.36 | 30.84 |
| Lactose Anhydrous | 38.165 | 305.32 | 76.33 |
| Microcrystalline | 38.165 | 305.32 | 76.33 |
| Cellulose | | | |
| Silicon Dioxide | 2.00 | 16.0 | 4.00 |
| Croscarmellose | 2.50 | 20.0 | 5.00 |
| sodium | | | |
| Magnesium Stearate | 0.50 | 4.0 | 1.00 |

Extra-granular

| | | | |
|---|---|---|---|
| Croscarmellose | 2.50 | 20.0 | 5.00 |
| sodium | | | |
| Magnesium Stearate | 0.75 | 6.0 | 1.50 |
| Total | 100.0 | 800.0 | 200.00 |
| Film Coat | 3.00 | 24.00 | 6.00 |
| Total | | 824.00 | 206.00 |

^{a}Strength indicated is as free base.

Comparative compositions containing croscarmellose sodium are shown in Table 3.

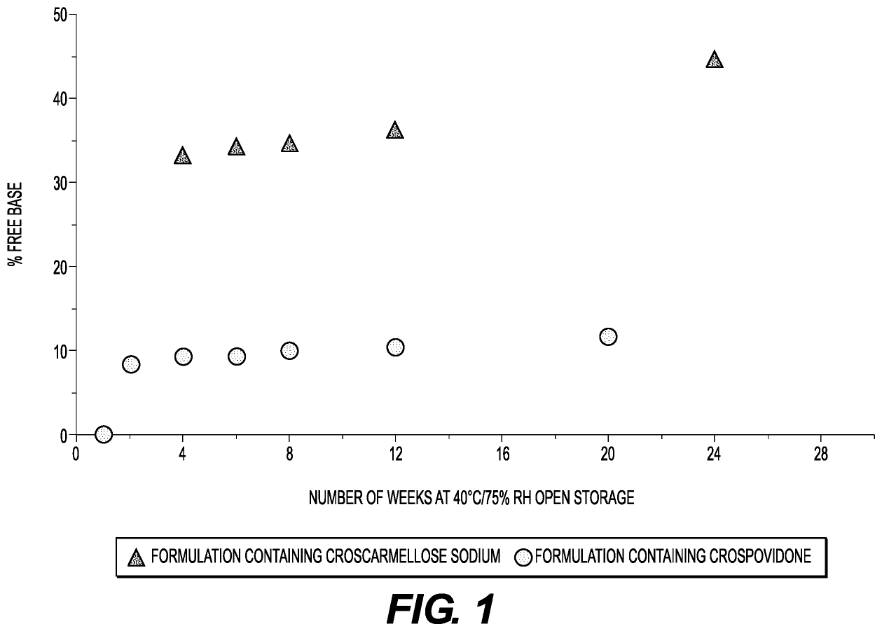

Compositions with croscarmellose sodium from Table 3 were then compared to compositions containing crospovidone from, for example, Table 1. The compositions were subjected to long term stability tests as illustrated in FIG. 1, which shows the levels of free base in the two tablet compositions containing croscarmellose sodium and crospovidone. Surprisingly, the composition containing crospovidone was found to have a much lower level of free base (12.1% when stored at 40° C./75% RH open for 24 weeks) compared to the composition containing croscarmellose sodium (45.7%).

Table 4 summarizes the level of free base observed upon storage at different conditions for up to 6 months. As shown in Table 4, the data confirmed that the addition of croscarmellose sodium into the composition can lead to a higher salt disproportionation upon storage.

Accordingly, crospovidone was used as the disintegrant instead of croscarmellose sodium for the final tablet composition. The results also confirmed that the tablet blends showed lower salt disproportionation than the coated tablets. At 24 weeks, 5 grams of the final blend stored in 200 mL HDPE bottles at 25° C./60% RH afforded less than 3% conversion to the free base.

TABLE 4
Percentage Free Base Level for Tablet Formulations
Containing Croscarmellose Sodium

| | | Time Point (Weeks) | | | |
|---|---|---|---|---|---|
| | | 0 | 4 | 12 | 24 |
| Packaging | Storage Conditions | % Free Base | | | |
| 5 grams final | 25° C./60% RH closed | NA | <3 | NA | <3 |
| blend in 200 cc | 40° C./75% RH closed | | 5.2 | NA | 9 |
| HDPE Bottle | 40° C. 75% RH open | | 18.5 | NA | 21.0 |
| 2 tablets in | 25° C./60% RH closed | NA | 5.2 | 5.6 | 7.7 |
| 200 cc | 40° C./75% RH closed | | 22.4 | 28.4 | 38.8 |
| HDPE Bottle | 40° C. 75% RH open | | 34.1 | 36.0 | 45.7 |
| 12 tablets in | 40° C./75% RH closed | | 13.4 | 14.0 | 29.3 |
| 200 cc | with desiccant | | | | |
| HDPE bottle | | | | | |

### Example 2: API/Magnesium Stearate Ratio

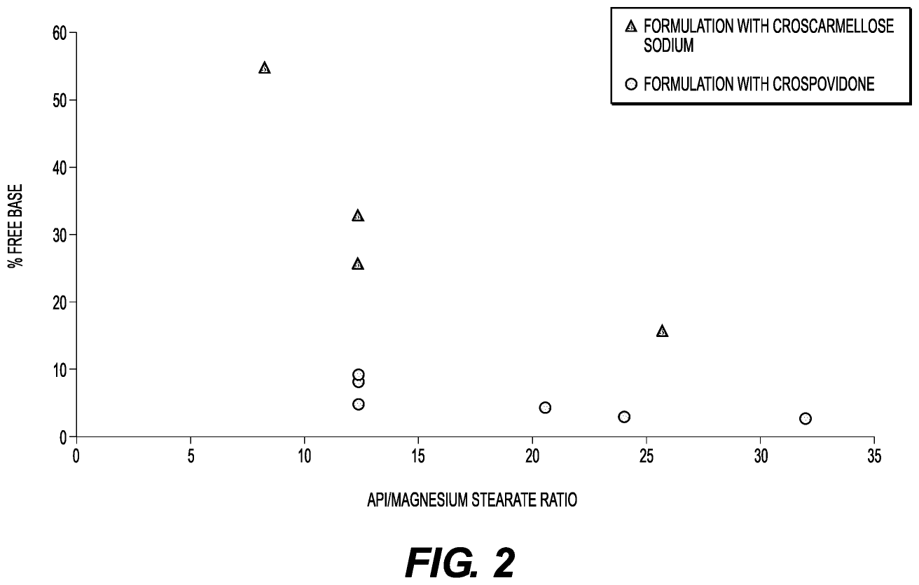

Referring to Table 5, the data demonstrates that the API/magnesium stearate ratio in the composition impacted the level of methane sulfonic acid salt disproportionation. FIG. 2 shows the % level of free base in tablet composition (or formulations) of different drug load/magnesium stearate ratios after 4 weeks of storage at 40° C./75% RH, open HDPE bottles. For compositions containing either croscarmellose sodium or crospovidone as the disintegrant, the level of free base decreased as the drug load/magnesium stearate ratio was increased (see Table 5). However, it was also observed that too high of a ratio of drug load/magnesium stearate increased processability parameters such as powder flow and sticking during roller compaction or during tableting. Thus, while a high ratio afforded better stability, a minimum amount of magnesium stearate was necessary.

TABLE 5
Formulation Details of Tablets Containing Croscarmellose Sodium or Crospovidone
and Drug Load/Magnesium Stearate Ratio, Acid Modifier or Different Lubricant

| | | | | | | | | |
|---|---|---|---|---|---|---|---|---|
| Formulation | A | B | C | D | E | F | G | H |

Intra-Granular (% w/w)

| | | | | | | | | |
|---|---|---|---|---|---|---|---|---|
| Compound I-MSA | 19.29 | 15.42 | 15.42 | 15.42 | 30.00 | 40.00 | 15.42 | 15.62 |
| Silicon dioxide | 2.00 | 2.00 | 2.00 | 2.00 | 2.00 | 2.00 | 2.00 | 2.02 |
| Microcrystalline | 36.48 | 56.33 | 38.165 | 38.165 | 30.875 | 25.875 | 37.165 | 38.65 |
| cellulose | | | | | | | | |
| Lactose anhydrous | 36.48 | 20.00 | 38.165 | 38.165 | 30.875 | 25.875 | 37.165 | 38.65 |
| Croscarmellose sodium | 2.50 | 2.50 | 2.50 | — | — | — | — | 2.53 |
| Crospovidone | — | — | — | 2.50 | 2.50 | 2.50 | 2.50 | — |
| Magnesium stearate | 0.50 | 0.75 | 0.75 | 0.75 | 0.75 | 0.75 | 0.75 | — |
| Citric acid | — | — | — | — | — | — | 2.00 | — |

Extra-Granular (% w/w)

| | | | | | | | | |
|---|---|---|---|---|---|---|---|---|
| Croscarmellose sodium | 2.50 | 2.50 | 2.50 | — | — | — | — | 2.53 |
| Crospovidone | — | — | — | 2.50 | 2.50 | 2.50 | 2.50 | — |
| Magnesium stearate | 0.25 | 0.50 | 0.50 | 0.50 | 0.50 | 0.50 | 0.50 | — |
| Stearic acid | — | — | — | — | 0.50 | 0.50 | — | — |
| Film Coat | — | — | 3.00 | 3.00 | — | — | — | — |
| Total (without coat) | 100.00 | 100.00 | 100.00 | 100.00 | 100.00 | 100.00 | 100.00 | 100.00 |
| Tablet type | Core | Core | Coated | Coated | Core | Core | Core | Core |
| API/Magnesium | 25.7 | 12.3 | 12.3 | 12.3 | 24.0 | 32.0 | 12.3 | — |
| stearate ratio | | | | | | | | |
| % free base after 8 | 20.50 | 32.10 | 34.60 | 10.00 | 5.00 | 5.00 | 6.00 | <3.00 |
| weeks @ 40° C./75% | | | | | | | | |
| RH in open storage | | | | | | | | |
| conditions | | | | | | | | |

### Example 4: Lubricant Study

In Table 6, the compositions containing both magnesium stearate and stearic acid were evaluated for its effect on salt disproportionation. A comparison of the compositions in Table 6 at 4 weeks at 40° C./75% RH open conditions demonstrated lower salt disproportionation (˜9% free base for magnesium stearate versus less than 3% for stearic acid) when stearic acid was used a lubricant. However, higher levels of stearic acid was found to be needed to provide similar levels of lubricity as magnesium stearate. When compositions containing magnesium stearate were protected from moisture and elevated temperatures, the compositions provided similar levels of salt disproportionation as the stearic acid compositions.

TABLE 6
Compositions with Magnesium Stearate
or Stearic Acid as Lubricant

| | | | | |
|---|---|---|---|---|
| | | Quantity per Tablet | | mg in |
| | % | mg in 100 mg | % | 100 mg |
| Component | w/w | tablets | w/w | tablets |

Intra-granular

| | | | | |
|---|---|---|---|---|
| Compound I-MSA | 15.42^{a} | 123.36 | 15.420^{a} | 123.36 |
| Lactose Anhydrous | 38.165 | 305.32 | 38.04 | 304.32 |
| Microcrystalline | 38.165 | 305.32 | 38.04 | 304.32 |
| Cellulose | | | | |
| Silicon Dioxide | 2.00 | 16.00 | 2.00 | 16.00 |
| Crospovidone | 2.50 | 20.0 | 2.50 | 20.00 |
| Magnesium Stearate | 0.50 | 4.00 | NA | NA |
| Stearic acid | NA | NA | 1.50 | 12.00 |

Extra-granular

| | | | | |
|---|---|---|---|---|
| Crospovidone | 2.50 | 20.0 | 2.50 | 20.00 |
| Magnesium Stearate | 0.75 | 6.00 | NA | NA |
| Stearic Acid | | | 1.00 | 8.00 |
| Total | 100.00 | 800.00 | 100.00 | 800.00 |

^{a}Strength as free base.

### Example 5: Effect of Surface Area on Stability

Two selected coated tablet formulations were subjected to stress storage conditions to study the difference of blends versus compacts. The data in Table 7 (see also Table 4) illustrates that the final blend was less prone to salt disproportionation indicating that the mechanical stress during compression contributed to the level of free base observed in tablets at an accelerated rate.

The percentage of free base data for both batches is summarized in Table 7.

TABLE 7
Results Showing Blend Having Better Stability Than Compacts

| | | Time Points (Weeks) | | | |
|---|---|---|---|---|---|
| | | % Free Base | | | |
| Packaging | Storage Conditions | 0 | 4 | 12 | 24 |
| 5 g final blend | 25° C./60% RH closed | NA | <3 | NA | <3 |
| in 200 cc HDPE | 40° C./75% RH closed | | 5.2 | NA | 9.0 |
| bottle | 40° C./75% RH open | | 18.5 | NA | 21.0 |
| 2 tablets in 200 | 25° C./60% RH closed | NA | 5.2 | 5.6 | 7.7 |
| cc HDPE bottle | 40° C./75% RH closed | | 22.4 | 28.4 | 38.8 |
| 12 tablets in 200 | 40° C./75% RH open | | 34.1 | 36.0 | 45.7 |
| cc HDPE bottle | 40° C./75% RH closed | | 13.4 | 14.0 | 29.3 |
| | with desiccant | | | | |

### Example 6: Effect of Particle Size on Stability

In a study of the effect of the particle size of Compound I-MSA on stability, the Compound I-MSA particle size to surface area ratio surprisingly did not have any effect on stability. Applicant measured the amount of free base at 40° C./75% RH open conditions after 12 weeks between the inventive composition containing unmilled API (larger particle size with D90 of 165 microns showed about 9.4% free base) versus milled API (smaller particle size with D90 of <20 microns showed about 10.4% free base). These results showed comparable amounts of salt disproportionation.

It is appreciated by one of skill in the art that fine materials (small particle size of the drug) are relatively more susceptible to stability issues from atmospheric oxygen, heat, light, humidity, and interacting excipients than larger or coarser particle sizes. In other words, it is known that active pharmaceutical ingredients with a smaller particle size show more disproportionation compared to ones with a large particle size. In the present application, no impact of particle size was observed in the range from about 7 microns to 165 microns. Accordingly, these results were surprising as the particle size to surface area ratio is known to have an impact on stability due to the higher surface area.

### Example 7: Stability Provided by Different Packaging

TABLE 8
SALT DISPROPORTIONATION IN
VARIOUS STORAGE CONDITIONS

| | | Time Points (Months) | | | |
|---|---|---|---|---|---|
| | | % Free Base | | | |
| Packaging | Storage Conditions | 0 | 1 | 3 | 6 |
| PVC/Aclar | 25° C./ 60% RH closed | NA | <3 | <3 | <3 |
| Blister | 30° C./75% RH closed | | <3 | <3 | <3 |
| | 40° C./75% RH closed | | <3 | 3.0 | 4.2 |
| Alu/Alu | 25° C./60% RH closed | NA | <3 | <3 | <3 |
| Blister | 30° C./75% RH closed | | <3 | <3 | <3 |
| | 40° C./75% RH closed | | <3 | <3 | <3 |
| 30 tablets in | 25° C./60% RH closed | NA | <3 | <3 | <3 |
| 200 cc HDPE | with desiccant | | | | |
| bottle | 30° C./75% RH closed | | <3 | <3 | <3 |
| | with desiccant | | | | |
| | 40° C./75% RH open | | 5.6 | 6.8 | 6.6 |
| | 40° C./75% RH closed | | <3 | <3 | <3 |
| | with desiccant | | | | |

As shown in Table 8, tablets packaged in closed high-density polyethylene (HDPE) bottles with desiccant and alu/alu (aluminum-aluminum foil blister) stored at 25° C./60% relative humidity (RH), 30° C./75% relative humidity, and 40° C./75% relative humidity for 6 months led to salt disproportion levels below the detection limit. The level of free base at 6 months in polyvinyl chloride/polychlortrifluorethylene (PVC/ACLARR) blisters was 4.2%.

The examples and embodiments described herein are for illustrative purposes only and in some embodiments, various modifications or changes are to be included within the purview of invention and scope of the appended claims.

What is claimed is:

1. A pharmaceutical composition suitable for oral administration comprising:
(i) a therapeutically effective amount of (R)—N-(4-chlorophenyl)-2-((1S, 4S)-4-(6-fluoroquinolin-4-yl) cyclohexyl) propanamide methane sulfonic acid salt present in an amount between 5% to 40% w/w of the composition having the structure: 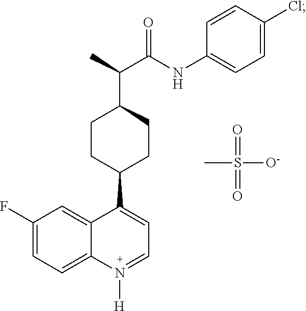
(ii) crospovidone as a disintegrant present in an amount between 2.0% to 7.0% w/w of the composition; and,
(iii) magnesium stearate as a lubricant present in amount between 0.25% to 1.75% w/w of the composition;
wherein the ratio of (R)—N-(4-chlorophenyl)-2-((1S, 4S)-4-(6-fluoroquinolin-4-yl) cyclohexyl) propanamide methane sulfonic acid salt to total magnesium stearate is 8.0 to 40.0 by weight; and,
wherein salt disproportionation of (R)—N-(4-chlorophenyl)-2-((1S,4S)-4-(6-fluoroquinolin-4-yl)cyclohexyl)propanamide methane sulfonic acid to (R)—N-(4-chlorophenyl)-2-((1S,4S)-4-(6-fluoroquinolin-4-yl)cyclohexyl)propanamide is less than 25% by weight.

2. The pharmaceutical composition of claim 1 further comprising microcrystalline cellulose as a first diluent and lactose anhydrous as a second diluent present in a total amount between 50% to 80% w/w of the composition.

3. The pharmaceutical composition of claim 1 further comprising silicon dioxide as a glidant present in an amount of 1.0% to 3.0% w/w of the composition.

4. The pharmaceutical composition of claim 1, wherein salt disproportionation of (R)—N-(4-chlorophenyl)-2-((1S, 4S)-4-(6-fluoroquinolin-4-yl) cyclohexyl) propanamide methane sulfonic acid to (R)—N-(4-chlorophenyl)-2-((1S, 4S)-4-(6-fluoroquinolin-4-yl) cyclohexyl) propanamide is less than 5% by weight.

5. The pharmaceutical composition of claim 1, wherein salt disproportionation of (R)—N-(4-chlorophenyl)-2-((1S, 4S)-4-(6-fluoroquinolin-4-yl) cyclohexyl) propanamide methane sulfonic acid to (R)—N-(4-chlorophenyl)-2-((1S, 4S)-4-(6-fluoroquinolin-4-yl) cyclohexyl) propanamide is less than 3% by weight.

6. The pharmaceutical composition of claim 1, wherein the ratio of (R)—N-(4-chlorophenyl)-2-((1S, 4S)-4-(6-fluoroquinolin-4-yl) cyclohexyl) propanamide methane sulfonic acid salt to total magnesium stearate is 23.0 to 40.0 by weight.

7. The pharmaceutical composition of claim 1, wherein the (R)—N-(4-chlorophenyl)-2-((1S, 4S)-4-(6-fluoroquinolin-4-yl) cyclohexyl) propanamide methane sulfonic acid salt present in an amount between 15% to 20% w/w of the composition.

8. The pharmaceutical composition of claim 2, wherein the first diluent and the second diluent is present in a ratio ranging from 2:1 to 1:2 by weight.

9. The pharmaceutical composition of claim 2, wherein the first diluent is present in amount ranging from 25% to 40% w/w of the composition.

10. The pharmaceutical composition of claim 2, wherein the second diluent is present in amount ranging from 25% to 40% w/w of the composition.

11. The pharmaceutical composition of claim 3, wherein the silicon dioxide is present in an amount of 2.0% w/w of the composition.

12. The pharmaceutical composition of claim 1, wherein the composition comprises an intra-granular phase and an extra-granular phase.

13. The pharmaceutical composition of claim 12, wherein the composition comprises:
(a) an intra-granular phase comprising:
(i) (R)—N-(4-chlorophenyl)-2-((1S, 4S)-4-(6-fluoroquinolin-4-yl) cyclohexyl) propanamide methane sulfonic acid salt present in an amount between 12% to 18% w/w of the composition;
(ii) crospovidone as a disintegrant present in an amount of 2% to 3% w/w of the composition;
(iii) magnesium stearate as a lubricant present in an amount of 0.25% to 0.75% w/w of the composition;
(b) an extra-granular phase comprising:
(i) crospovidone as a disintegrant present in an amount of 2% to 3% w/w of the composition; and,
(ii) magnesium stearate as a lubricant present in an amount of 0.50% to 1.00% w/w of the composition.

14. The pharmaceutical composition of claim 13, wherein the intra-granular phase further comprises microcrystalline cellulose as a first diluent and lactose anhydrous as a second diluent present in a total amount between 75% to 80% w/w of the composition; and, silicon dioxide as a glidant present in an amount of 1.5% to 2.5% w/w of the composition.

15. The pharmaceutical composition of claim 1, wherein salt disproportionation of (R)—N-(4-chlorophenyl)-2-((1S,4S)-4-(6-fluoroquinolin-4-yl)cyclohexyl)propanamide methane sulfonic acid to (R)—N-(4-chlorophenyl)-2-((1S,4S)-4-(6-fluoroquinolin-4-yl)cyclohexyl)propanamide is less than 10% by weight after 12 weeks at 40° C. and 75% relative humidity and has a particle size distribution characterized by a D90 has a value from about 7 microns to about 165 microns.

16. The pharmaceutical composition of claim 1, wherein salt disproportionation of (R)—N-(4-chlorophenyl)-2-((1S,4S)-4-(6-fluoroquinolin-4-yl)cyclohexyl)propanamide methane sulfonic acid to (R)—N-(4-chlorophenyl)-2-((1S,4S)-4-(6-fluoroquinolin-4-yl)cyclohexyl)propanamide is less than 3% by weight after 24 weeks stored in a 200 cc high-density polyethylene bottle at 25° C. and 60% relative humidity.

17. The pharmaceutical composition of claim 1, wherein the particle size distribution characterized by a D90 has a value from about 10 microns to about 165 microns.

18. The pharmaceutical composition of claim 1, wherein salt disproportionation of (R)—N-(4-chlorophenyl)-2-((1S,4S)-4-(6-fluoroquinolin-4-yl)cyclohexyl)propanamide methane sulfonic acid to (R)—N-(4-chlorophenyl)-2-((1S,4S)-4-(6-fluoroquinolin-4-yl)cyclohexyl)propanamide is less than 3% by weight after 6 months at 25° C. and 60% relative humidity with blister packaging.

19. The pharmaceutical composition of claim 1, wherein salt disproportionation of (R)—N-(4-chlorophenyl)-2-((1S,4S)-4-(6-fluoroquinolin-4-yl)cyclohexyl)propanamide methane sulfonic acid to (R)—N-(4-chlorophenyl)-2-((1S,4S)-4-(6-fluoroquinolin-4-yl)cyclohexyl)propanamide is less than 3% by weight after 4 weeks at 25° C. and 60% relative humidity.

20. The pharmaceutical composition of claim 1, wherein salt disproportionation of (R)—N-(4-chlorophenyl)-2-((1S,4S)-4-(6-fluoroquinolin-4-yl)cyclohexyl)propanamide methane sulfonic acid to (R)—N-(4-chlorophenyl)-2-((1S,4S)-4-(6-fluoroquinolin-4-yl)cyclohexyl)propanamide is less than 3% by weight after 4 weeks at 40° C. and 75% relative humidity.

21. The pharmaceutical composition of claim 1, wherein the composition is a blend and the salt disproportionation of (R)—N-(4-chlorophenyl)-2-((1S,4S)-4-(6-fluoroquinolin-4-yl)cyclohexyl)propanamide methane sulfonic acid to (R)—N-(4-chlorophenyl)-2-((1S,4S)-4-(6-fluoroquinolin-4-yl)cyclohexyl)propanamide is less than 3% by weight after 24 weeks at 25° C. and 60% closed relative humidity.

22. The pharmaceutical composition of claim 1, wherein the composition is selected from the group consisting of tablet, crushed tablet, capsule or sprinkled contents from a capsule, mini-tablets, and beads.

23. The pharmaceutical composition of claim 1 further comprising citric acid.
